(19) United States
(12) Patent Application Publication
(10) Pub. No.: US 2022/0193392 A1
(43) Pub. Date: Jun. 23, 2022

(54) Facilitate Delivery of Devices with High Friction - Braid

(71) Applicant: Boston Scientific Scimed Inc., Maple Grove, MN (US)

(72) Inventors: Qian Liu, Plymouth, MN (US); Brian R. Reynolds, Ramsey, MN (US); Timothy Rubesch, Blaine, MN (US)

(21) Appl. No.: 17/560,029

(22) Filed: Dec. 22, 2021

Related U.S. Application Data
(60) Provisional application No. 63/130,220, filed on Dec. 23, 2020.

(51) Int. Cl. A61M 60/865; A61M 60/178

(52) U.S. Cl. CPC A61M 60/865; A61M 60/178

(57) ABSTRACT

A delivery device for percutaneous medical device is disclosed herein. The delivery device includes an enveloper member that includes an enveloper wall, an enveloper aperture that is formed at the enveloper wall such that the enveloper member is configured to receive a deployment portion of the percutaneous medical device through the enveloper aperture, and a tether assembly extending from the enveloper wall; the enveloper member configured to removably cover the deployment portion with the enveloper wall; and the tether assembly configured to facilitate removal of the enveloper member from covering the deployment portion and to facilitate withdrawal of the enveloper member from a delivery lumen.

### CROSS-REFERENCE TO RELATED APPLICATIONS

This application claims priority to Provisional Application No. 63/130,220, filed Dec. 23, 2020, which is herein incorporated by reference in its entirety.

### TECHNICAL FIELD

The present disclosure relates to delivery aids for percutaneous circulatory support devices. More specifically, the present disclosure relates to delivery aides for integrated braided cannulas in a circulatory support pump.

### BACKGROUND

Circulatory support devices support the pumping action of the heart. These devices may be disposed through a valve opening such as, for example, an aortic valve. Often, circulatory support devices have a polymeric coating, which for example protects underlying components such as a mesh of the circulatory support devices. Such coatings, however, can make the circulatory support devices resistive to travel through lumens used for delivering such devices to treatment sites.

### SUMMARY

In Example 1, a delivery device for percutaneous medical device comprises an enveloper member and a tether assembly, the enveloper member that includes an enveloper wall, an enveloper aperture that is formed at the enveloper wall such that the enveloper member is configured to receive a deployment portion of the percutaneous medical device through the enveloper aperture, and a tether assembly extending from the enveloper wall; the enveloper member configured to removably cover the deployment portion with the enveloper wall; and the tether assembly configured to facilitate removal of the enveloper member from covering the deployment portion and to facilitate withdrawal of the enveloper member from a delivery lumen.

In Example 2, the delivery device of Example 1, wherein the tether assembly is fixedly connected to the enveloper member.

In Example 3, the delivery device as in one of Example 1 and 2, wherein the tether assembly includes a collar that connects the tether assembly to the enveloper member; wherein the collar is optionally configured to inhibit motion of the enveloper member relative to the percutaneous medical device.

In Example 4, the delivery device as in one of Examples 1-3, wherein a distal portion of the enveloper member includes a securement member that is configured to inhibit expansion of the distal portion of the enveloper member when the securement member is secured and to allow the distal portion of the enveloper member to expand when the securement member is released; and wherein the securement member optionally comprises silicone.

In Example 5, the delivery device as in one of Examples 1-4, wherein the enveloper member is a braided mesh enveloper member; and wherein the enveloper member optionally comprises nitinol.

In Example 6, a delivery assembly for a percutaneous medical device includes a transfer tube and a delivery device, the transfer tube having an inner lumen that is configured to removably house a deployment portion of the percutaneous medical device; and the delivery device comprising: an enveloper member that includes an enveloper wall, an enveloper aperture that is formed at the enveloper wall such that the enveloper member is configured to receive a deployment portion of the percutaneous medical device through the enveloper aperture, and a tether assembly extending from the enveloper wall, the enveloper member configured to removably cover the deployment portion with the enveloper wall, and the tether assembly configured to facilitate withdrawal of the enveloper member from covering the deployment portion and to facilitate withdrawal of the enveloper member from a delivery lumen.

In Example 7, the delivery assembly of Example 6, wherein the tether assembly is fixedly connected to the enveloper member.

In Example 8, the delivery assembly as in one of Examples 6-7, wherein the tether assembly includes a collar that connects the tether assembly to the enveloper member at a proximal end of the enveloper member; and wherein the collar is optionally configured to inhibit motion of the enveloper member relative to the percutaneous medical device.

In Example 9, the delivery assembly as in one of Examples 6-8, wherein a distal portion of the enveloper member includes a securement member that is configured to inhibit expansion of the distal portion of the enveloper member when the securement member is secured and to allow the distal portion of the enveloper member to expand when the securement member is released.

In Example 10, the delivery assembly as in one of Examples 6-9, wherein the enveloper member is a braided mesh enveloper member; and wherein the securement member optionally comprises silicone.

In Example 11, the delivery assembly as in one of Examples 6-10, wherein the enveloper member comprises nitinol.

In Example 12, a method of delivering a percutaneous medical device includes surrounding a deployment portion of the percutaneous medical device with a delivery device comprising: an enveloper member that includes an enveloper wall, an enveloper aperture that is formed at the enveloper wall such that the enveloper member is configured to receive a deployment portion of the percutaneous medical device through the enveloper aperture, the enveloper member configured to removably cover the deployment portion with the enveloper wall, and the tether assembly configured to facilitate withdrawal of the enveloper member from covering the deployment portion and to facilitate withdrawal of the enveloper member from a delivery lumen; inserting the deployment portion together with the enveloper member into a body lumen; and withdrawing the enveloper member from the body lumen.

In Example 13, the method as in Example 12 further comprising withdrawing the deployment portion together with the enveloper member into a transfer tube, and wherein inserting the deployment portion includes inserting the transfer tube into the body lumen and moving the deployment portion distally relative to the transfer tube such that the deployment portion is outside of the transfer tube.

In Example 14, the method as in one of Examples 12-13, wherein a distal portion of the enveloper member includes a securement member that is configured to inhibit expansion of the distal portion of the enveloper member when the securement member is secured and to allow the distal portion of the enveloper member to expand when the securement member is released, and wherein the tether assembly is configured to cause the securement member to transition from a secured state to a unsecured state, wherein inserting the deployment portion together with the enveloper member into the body lumen occurs with the enveloper member in the secured state, and wherein the method further comprises causing a portion of the enveloper member to transition from the secured state to the unsecured state.

In Example 15, the method as in one of Examples 12-14, wherein the tether assembly includes a collar that connects the tether assembly to the enveloper member at a proximal end of the enveloper member, and wherein the enveloper aperture is distally positioned relative to the collar.

In Example 16, a delivery device for percutaneous medical device includes an enveloper member and a tether assembly, the enveloper member that includes an enveloper wall, an enveloper aperture that is formed at the enveloper wall such that the enveloper member is configured to receive a deployment portion of the percutaneous medical device through the enveloper aperture, and the tether assembly extending from the enveloper wall; the enveloper member configured to removably cover the deployment portion with the enveloper wall; and the tether assembly configured to facilitate removal of the enveloper member from covering the deployment portion and to facilitate withdrawal of the enveloper member from a delivery lumen.

In Example 17, the delivery device of Example 16, wherein the tether assembly is fixedly connected to the enveloper member.

In Example 18, the delivery device of Example 16, wherein the tether assembly includes a collar that connects the tether assembly to the enveloper member.

In Example 19, the delivery device of Example 18, wherein the collar is configured to inhibit motion of the enveloper member relative to the percutaneous medical device.

In Example 20, the delivery device of Example 16, wherein a distal portion of the enveloper member includes a securement member that is configured to inhibit expansion of the distal portion of the enveloper member when the securement member is secured and to allow the distal portion of the enveloper member to expand when the securement member is released.

In Example 21, the delivery device of Example 20, wherein the enveloper member is a braided mesh enveloper member.

In Example 22, the delivery device of Example 21, wherein the securement member comprises silicone.

In Example 23, the delivery device of Example 16, wherein the enveloper member comprises nitinol.

In Example 24, a delivery assembly for a percutaneous medical device includes a transfer tube and a delivery device, the transfer tube having an inner lumen that is configured to removably house a deployment portion of the percutaneous medical device; and the delivery device comprising: an enveloper member that includes an enveloper wall, an enveloper aperture that is formed at the enveloper wall such that the enveloper member is configured to receive a deployment portion of the percutaneous medical device through the enveloper aperture, and a tether assembly extending from the enveloper wall, the enveloper member configured to removably cover the deployment portion with the enveloper wall, and the tether assembly configured to facilitate withdrawal of the enveloper member from covering the deployment portion and to facilitate withdrawal of the enveloper member from a delivery lumen.

In Example 25, the delivery assembly of Example 24, wherein the tether assembly is fixedly connected to the enveloper member.

In Example 26, the delivery assembly of Example 24, wherein the tether assembly includes a collar that connects the tether assembly to the enveloper member at a proximal end of the enveloper member.

In Example 27, the delivery assembly of Example 26, wherein the collar is configured to inhibit motion of the enveloper member relative to the percutaneous medical device.

In Example 28, the delivery assembly of Example 24, wherein a distal portion of the enveloper member includes a securement member that is configured to inhibit expansion of the distal portion of the enveloper member when the securement member is secured and to allow the distal portion of the enveloper member to expand when the securement member is released.

In Example 29, the delivery assembly of Example 28, wherein the enveloper member is a braided mesh enveloper member.

In Example 30, the delivery assembly of Example 29, wherein the securement member comprises silicone.

In Example 31, the delivery assembly of Example 24, wherein the enveloper member comprises nitinol.

In Example 32, a method of delivering a percutaneous medical device includes surrounding a deployment portion of the percutaneous medical device with a delivery device comprising: an enveloper member that includes an enveloper wall, an enveloper aperture that is formed at the enveloper wall such that the enveloper member is configured to receive a deployment portion of the percutaneous medical device through the enveloper aperture, the enveloper member configured to removably cover the deployment portion with the enveloper wall, and the tether assembly configured to facilitate withdrawal of the enveloper member from covering the deployment portion and to facilitate withdrawal of the enveloper member from a delivery lumen; inserting the deployment portion together with the enveloper member into a body lumen; and withdrawing the enveloper member from the body lumen.

In Example 33, the method of Example 32 further comprising withdrawing the deployment portion together with the enveloper member into a transfer tube, and wherein inserting the deployment portion includes inserting the transfer tube into the body lumen and moving the deployment portion distally relative to the transfer tube such that the deployment portion is outside of the transfer tube.

In Example 34, the method of Example 32, wherein a distal portion of the enveloper member includes a securement member that is configured to inhibit expansion of the distal portion of the enveloper member when the securement member is secured and to allow the distal portion of the enveloper member to expand when the securement member is released, and wherein the tether assembly is configured to cause the securement member to transition from a secured state to a unsecured state, wherein inserting the deployment portion together with the enveloper member into the body lumen occurs with the enveloper member in the secured state, and wherein the method further comprises causing a portion of the enveloper member to transition from the secured state to the unsecured state.

In Example 35, the method of Example 34, wherein the tether assembly includes a collar that connects the tether assembly to the enveloper member at a proximal end of the enveloper member, and wherein the enveloper aperture is distally positioned relative to the collar.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1A depicts a conceptual diagram of a circulatory support device including a cannula and an adaptor, according to aspects of the present disclosure.

FIG. 1B depicts a side view of the circulatory support device depicted in FIG. 1A, including a pump, according to aspects of the present disclosure.

FIG. 2 depicts a sideview of a delivery assembly, according to aspects of the present disclosure.

FIG. 3A depicts an enveloper member, according to aspects of the present disclosure.

FIG. 3B depicts the enveloper member in FIG. 3A positioned around a deployment portion, according to aspects of the present disclosure.

FIG. 3C depicts a second example of an enveloper member 220, according to aspects of the present disclosure.

FIG. 4 depicts a flowchart of a method, according to aspects of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Included in the present disclosure are circulatory support devices that have a decreased delivery force in comparison to conventional embodiments. For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

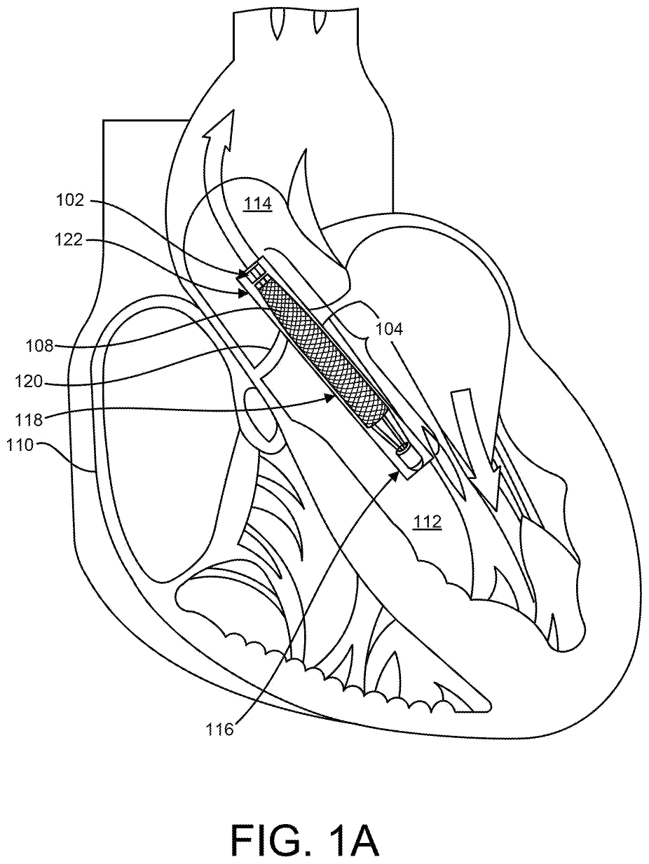

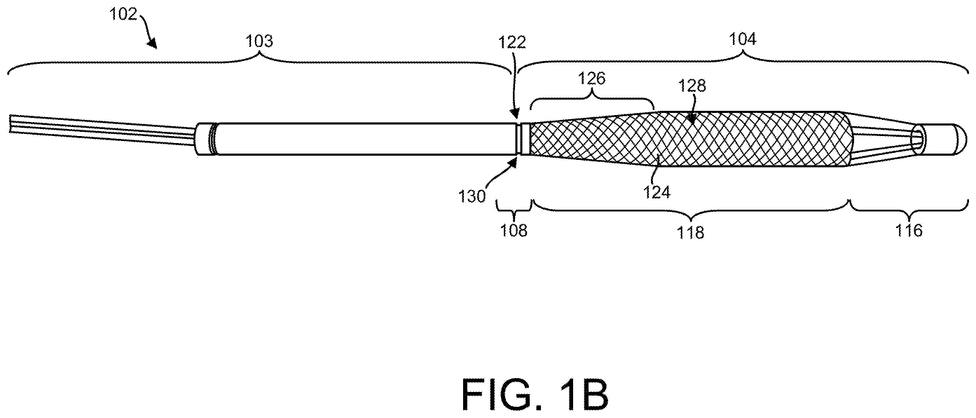

FIG. 1A depicts a conceptual diagram of a circulatory support device 102 including a cannula 104 and an adaptor 108, in accordance with embodiments of the subject matter disclosed herein. The circulatory support device 102 is shown arranged within a heart 110. According to embodiments, the circulatory support device 102 may include a ventricular assist device (shown in FIG. 1B), such as a pump, that is coupled to the cannula 104 by the adaptor 108. The ventricular assist device is configured to pump blood from the subject's left ventricle 112 into the subject's aorta 114. In embodiments, the circulatory support device 102 may be used to treat cardiogenic shock and other heart failure modalities.

In embodiments, a distal portion 116 of the circulatory support device 102 is arranged in the left ventricle 112. An intermediate portion 118 of the circulatory support device 102 extends through the aortic valve 120 so that a proximal portion 122 of the cannula 104 extends into the aorta 114. In embodiments, the proximal portion 122 of the cannula 104 is coupled to the adaptor 108 and the adaptor 108 is coupled to the circulatory support device 102. During operation, the circulatory support device 102 draws blood from the left ventricle 112, through the cannula 104 of the circulatory support device 102 and is released into the aorta 114. Additionally, or alternatively, the circulatory support device 102 may be used to facilitate pumping blood from some other aspect of the subject's vasculature into an adjacent portion of the vasculature.

FIG. 1B depicts a side view of the circulatory support device 102 depicted in FIG. 1A including the ventricular assist device 103, in accordance with embodiments of the subject matter disclosed herein.

As stated above, the cannula 104 may include a proximal portion 122, an intermediate portion 118, and a distal portion 116. The intermediate portion 118 may include a braided mesh 124 that extends between the proximal portion 122 and the distal portion 116. In embodiments, the braided mesh 124 may have various braid angles and/or varying braid angles, as explained in more detail below. In embodiments, a proximal portion 126 of the braided mesh 124 may be tapered. The tapered proximal portion 126 may transition the braided mesh 124 from a larger diameter (e.g., greater than or equal to 5 millimeters (mm)) near a distal end 128 of the proximal portion 126 to a smaller diameter near a proximal end 130 of the braided mesh 124. In embodiments, the braided mesh 124 may be collapsed into a smaller diameter for delivery into the heart 110. Once arranged within the heart 110, the braided mesh 124 may be expanded to its larger diameter. By being able to expand to a larger diameter than its delivery configuration, the cannula 104 may provide larger flow rates than can be provided with a non-expandable smaller diameter cannula 104. In embodiments, the braided mesh 124 may be designed to adequately withstand the pressure gradient between the inside and the outside of the cannula 104.

In embodiments, the braided mesh 124 is coated with a membrane to form a conduit through the cannula 104 from the distal portion 116 to the proximal portion 122. In embodiments, the membrane may be silicone. In embodiments, the cannula 104 is formed from a plurality of nitinol wires having a diameter of 0.008″. However, this is only an example and other types of wires having other diameters may be used to form the cannula 104. Additionally, or alternatively, wires having varying diameters may be used to form the cannula 104. In embodiments, the cannula 104 may be formed from a range of nitinol wires (e.g., 6 wires to 48 wires). While protective, the membrane coated over the braided mesh 124 can have a fairly resistive exterior that can prove troublesome during delivery of the circulatory support device 102, for example, by increasing the delivery force required to advance the cannula 104 through a delivery lumen 206 during setup and/or in operation.

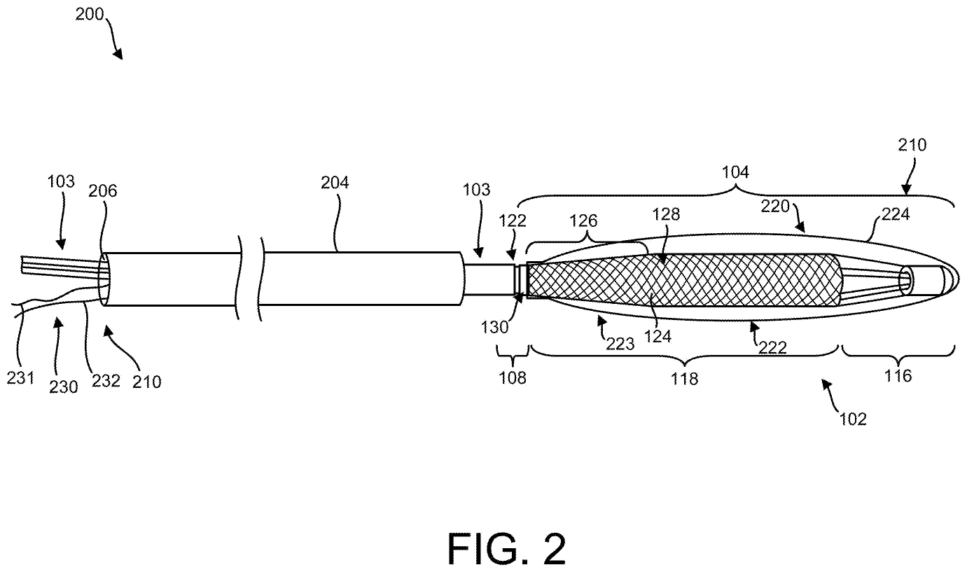

Turning to FIG. 2, a delivery assembly 200 for a percutaneous medical device 102, such as the circulatory support device 102 is shown. The percutaneous medical device 102 includes a deployment portion 104, such as the cannula 104. For illustration purposes, the proximal direction is in the direction from right to left on the figure, and the distal direction is in the direction from left to right on the figure. Further, hereinafter, for ease of illustration, the percutaneous medical device 102 will be generally referred to as 102, and the deployment portion 104 will be generally referred to as 104. But it should be noted that using the delivery assembly 200 with other percutaneous medical devices 102 and their deployment portions 104 is also contemplated and is therefore not outside the scope of this disclosure.

Considering the resistive nature of the membrane, the delivery assembly 200 can include an introducer 204, a delivery lumen 206 formed in the introducer 204, and a delivery device 210 to aide in delivery of the percutaneous medical device 102. The delivery lumen 206 can be configured to removably house a deployment portion 104 of the percutaneous medical device 102. For example, the introducer 204 can house the deployment portion 104 until it is inserted into a patient's vasculature. In some examples, the delivery lumen 206 is configured to constrain (e.g., by having a smaller diameter than) the deployment portion 104 or the combination of the deployment portion 104 and delivery device 210. In other examples, the delivery lumen 206 does not constrain the deployment portion 104 or the combination of the deployment portion 104 and delivery device 210. The delivery device 210 can include an enveloper member 220 and a tether assembly 230. The enveloper member 220 can be configured to removably cover the deployment portion 104 with the enveloper wall 222. The tether assembly 230 can be configured to facilitate withdrawal of the enveloper member 220 from covering the deployment portion 104 and to facilitate withdrawal of the enveloper member 220 from a delivery lumen 206.

As noted throughout, variations of the present, disclosure are contemplated. For instance, although shown and discussed herein as involving a introducer 204, it is contemplated, and thus not outside the scope of this disclosure, that some examples of the delivery assembly 200 may not include an introducer 204 or may instead include a differently shaped or formed member that functions similarly to the introducer 204. As well, although depicted as being closed at both ends, the enveloper member 220 can have one end open and the other end closed, or both ends open. Even these examples are just some of those which are contemplated and that would be appreciated by the skilled artisan when armed with this disclosure.

A resistance to insertion of the delivery device 210 through the delivery lumen 206 can be defined as a delivery force. The delivery force can be a function of an insertion depth such that the delivery force is highest at initial insertion or shortly thereafter and lowest when close to delivery. As noted above, the percutaneous medical device 102 has a fairly resistive exterior in the membrane, which results in a high delivery force. Traditional measures for reducing the delivery force include lubricating and/or wetting one or more portions of the delivery assembly 200, such as the cannula 104, the introducer 204, or both. These measures, however, may still result, in a relatively high delivery force that is undesirable. In examples, the enveloper member 220 can provide a lower delivery force than that of the percutaneous medical device 102. As well, the delivery force provided by the delivery device 210 can be lower than that of traditional lubricating measures, for example. In this regard, the delivery force can be reduced by as much as approximately 4× in some examples and on the order of approximately 3×, 2.5×, 2×, 1.5×, etc. in other examples.

When positioned around a portion of the deployment portion 104, the enveloper member 220 can reduce delivery forces required to deliver the deployment portion 104 through the delivery lumen 206. The enveloper member 220 can include an enveloper wall 222, an enveloper aperture 223 that is formed at the enveloper wall 222 such that the enveloper member 220 is configured to receive a deployment portion 104 of the percutaneous medical device 102 through the enveloper aperture 223, and a tether assembly 230 extending from the enveloper wall 222. During setup, the deployment portion 104 can be received in the enveloper aperture 223 (e.g., positioned at a distal portion, a proximal portion, or somewhere therebetween) so as to have a portion (e.g., all) of the deployment portion 104 positioned within the enveloper member 220. So configured, the deployment portion 104 together with the enveloper member 220 can be inserted, with a reduced delivery force, into the delivery lumen 206. Under these circumstances, the enveloper member 220 can be conformable to the deployment portion 104 and to the delivery lumen 206. As discussed further below, in examples, the tether assembly 230 can be used to withdraw (e.g., via proximal movement of the tether assembly 230) only the enveloper member 220 from the delivery lumen 206 prior to deployment of the deployment portion 104. Under these circumstances, the enveloper member 220 moves from overlaying or being positioned around a portion of the deployment portion 104 to being withdrawn (e.g., distally moved relative to the deployment portion 104) to thereby reveal the deployment portion 104.

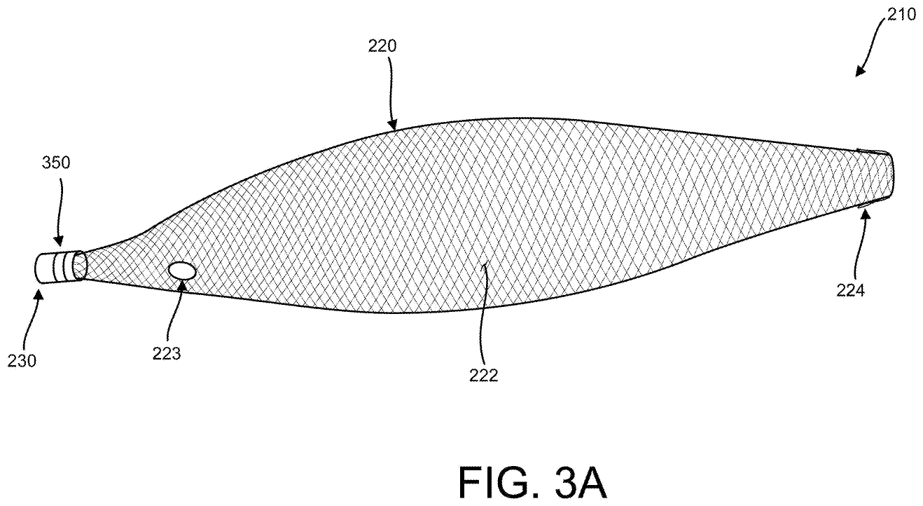

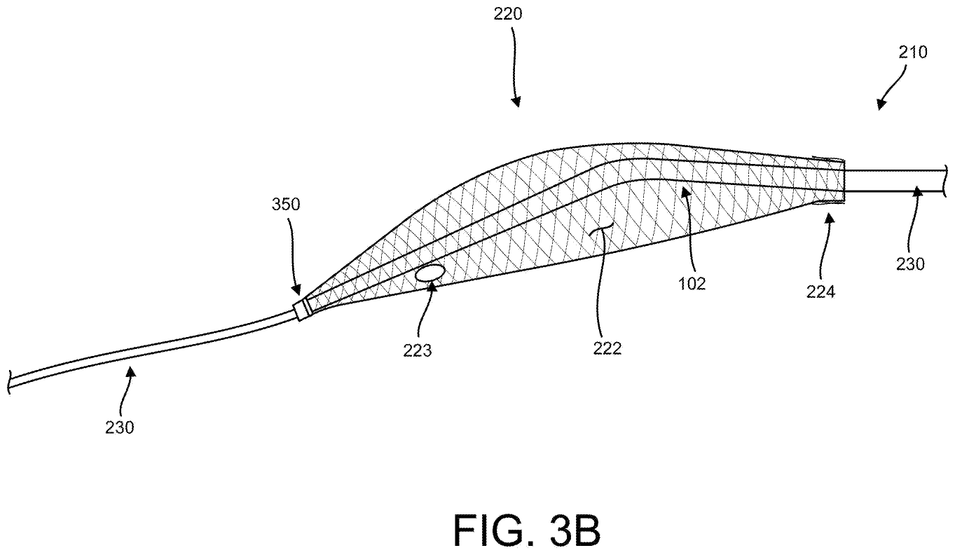

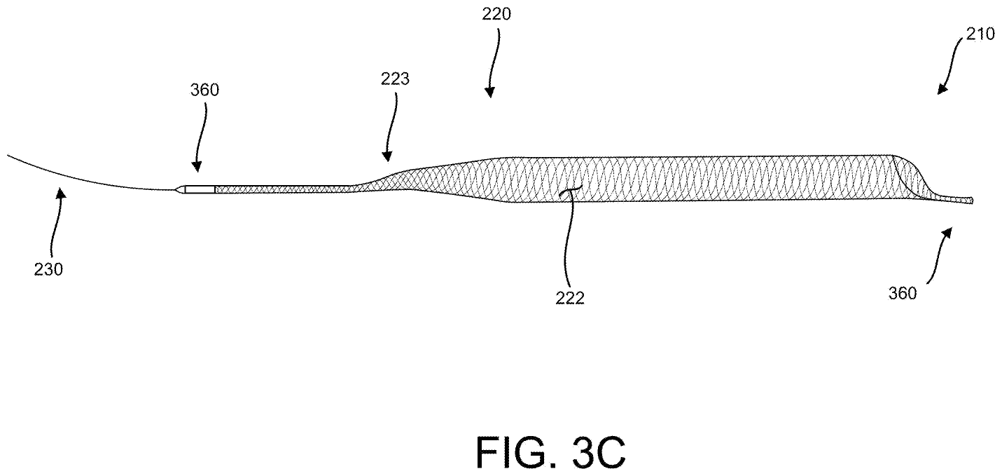

FIGS. 3A and 3B show various views of the delivery device 210, according to aspects of the present disclosure. FIG. 3A depicts a first example of an enveloper member 220. FIG. 3B depicts the enveloper member 220 in FIG. 3A positioned around a deployment portion 104. FIG. 3C depicts a second example of an enveloper member 220.

In examples, as is shown here in these figures, the enveloper member 220 is a braided mesh enveloper member 220 made from a generally flexible material. In examples, the enveloper member 220 comprises nitinol (e.g., a plurality of nitinol strands) or other similar material around an interior space, which can be large enough to house the deployment portion 104. In this regard, the braided mesh enveloper member 220 can be a morphable, flexible structure such that manipulation at one portion of the braided mesh enveloper member 220 can cause manipulation of another portion of the braided mesh enveloper member 220. As an example, the braided mesh enveloper member 220 can begin as a generally tubular structure with opposing ends and be manipulated at the opposing ends such that a middle portion of the braided mesh enveloper member 220 balloons in diameter while the opposing end are reduced in diameter. Under these circumstances, a longitudinal cross section (e.g., taken along the proximal and distal directions) of the braided mesh enveloper member 220 can be generally ellipsoidal in shape. The result can be a braided mesh enveloper member 220 that is relatively loose-fitting over the deployment portion 104 but, for example, not so much so as to cause bunching during insertion of the braided mesh enveloper member 220 together with the deployment portion 104 into the delivery lumen 206. Other types of cross sections (e.g., eccentric, symmetrical, etc.), corresponding manipulations, and corresponding fits (e.g., constraint-type fit) over the deployment portion 104 are contemplated and should not be considered outside the scope of this disclosure.

A resilient portion can secure the braided mesh enveloper member 220 over the deployment portion 104. In examples, a distal portion of the enveloper member 220 can include a securement member 224 that is configured to inhibit expansion of the distal portion of the enveloper member 220 when the securement member 224 is secured and to allow the distal portion of the enveloper member 220 to expand when the securement member 224 is released. In examples, the securement member 224 can comprise silicone or similar material. In this regard, the securement member 224 can be a resilient portion of the braided mesh enveloper member 220 and can have a level of resilience (e.g., radial resilience) that withstands the delivery force so that the enveloper member 220 is inhibited from inadvertently expanding and thereby prematurely releasing the deployment portion 104 from the enveloper member 220.

Construction of the delivery device 210 can facilitate accommodation of various forces (e.g., delivery forces and grabbing forces) experienced by the delivery device 210 during operation. In examples, a length of the tether assembly 230 can be longer than a length of the introducer 204 such that a grabbable amount of the tether assembly 230 remains protruding from the proximal end of the introducer 204 as the enveloper member 220 is advanced through the introducer 204 and to a deployment site where the deployment portion 104 is deployed. The tether assembly 230 can be constructed of one or multiple materials, each of which is of sufficient rigidity to withstand the delivery force experienced by the enveloper member 220, the deployment portion 104 being inserted (e.g., withdrawn) into the enveloper member 220, and/or a grabbing force on the grabbable amount. The grabbing force can be the force required to cause the securement member 224 to expand. Although in some instances, the grabbing force may be greater than the delivery force, in many instances, the grabbing force may be less than or equal to the delivery force.

In examples, the tether assembly 230 can be integrally manufactured with or a separate component that is connected to the enveloper member 220. When a separate component from the enveloper member 220, the tether assembly 230 can be (e.g., fixedly or rigidly) connected to the enveloper member 220 in various ways. As seen in FIGS. 3A and 3B, in a first example, the tether assembly 230 can include a collar 350 that connects the tether assembly 230 to the enveloper member 220 (e.g., along the enveloper wall 222 or at the distal or proximal portions of the enveloper member 220). This connection can be relatively rigid or semi-rigid in nature. In examples, the collar 350 can be configured to inhibit motion of the enveloper member 220 relative to the percutaneous medical device 102. For example, the deployment portion 104 (or neighboring portions at a distal end of a catheter where the deployment portion 104 is mounted) can have a first diameter and the collar 350 can have a second diameter that is smaller than the first diameter. As seen in FIG. 3C, in a second example the tether assembly 230 can be connected to the enveloper member 220 via an adhesive cap 360, which can optionally cause the enveloper member to be parted to one side such that the enveloper member tapers from the distal portion to the proximal portion thereof. Note that an adhesive cap 360 can optionally be provided on the distal portion of the enveloper member thereby making whichever ends include the adhesive cap atraumatic. With the connection to between the enveloper member 220 and tether assembly 230 so configured (e.g., as shown in FIGS. 3A-3C), the enveloper member can be inhibited from traveling past a proximal end of the deployment portion 104. Under these circumstances, the enveloper member 220 can be temporally inhibited from moving relative to the deployment portion 104 (e.g., until the grabbing force is applied to the tether assembly 230).

During operation, the tether assembly 230 can be configured to cause the securement member 224 to actuate between the secured and unsecured states. For example, the tether assembly 230 can cause the securement member 224 to actuate from the secured state when the percutaneous medical device 102 is being delivered to the unsecured state prior to when the deployment portion 104 of the percutaneous medical device 102 is deployed. For instance, as discussed in further detail below, the tether assembly 230 can cause the securement member 224 to expand by pulling on the tether assembly 230 in the proximal direction. In this regard, the securement member 224 can be brought into abutment with the deployment portion 104. Under these circumstances, a radially outward force as the securement member 224 is gradually moved along a portion of the deployment portion 104 can aide in expanding the securement member 224.

Additionally, or alternatively, the tether assembly 230 can be configured to facilitate withdrawal of the enveloper member 220 from overlaying the deployment portion 104 and/or from the delivery lumen 206. For example, pulling on the tether assembly 230 in the proximal direction can cause proportional movement of the enveloper member 220. To begin, the enveloper member 220 can be delivered while overlaying the deployment portion 104 (e.g., in the constrained configuration). When the enveloper member 220 is in the expanded configuration just before the deployment portion 104 is deployed, the tether assembly 230 can be pulled using the grabbing force to thereby facilitate the enveloper member 220 moving proximally so as to no longer overlay the deployment portion 104. Then, in addition or in alternative, the tether assembly 230 can be pulled using the grabbing force to thereby facilitate the withdrawal of the enveloper member 220 from the delivery lumen 206.

While a single tether in some example, the tether assembly 230 in other examples can include a plurality of tethers. For instance, the plurality of tethers can include a first tether and a second tether. The first tether can be configured to actuate the securement member 224 from the secured state to the unsecured state. The second tether can be configured to facilitate withdrawal of the enveloper member 220 from overlaying the deployment portion 104 and to facilitate withdrawal of the enveloper member 220 from the delivery lumen 206. In some examples, the first and second tethers 231, 232 can comprise different materials, each of which are better suited for their particular purpose. For example, the first tether can comprise a more rigid material than the second tether or vice versa. In other examples, the first and second tether can comprise approximately the same material.

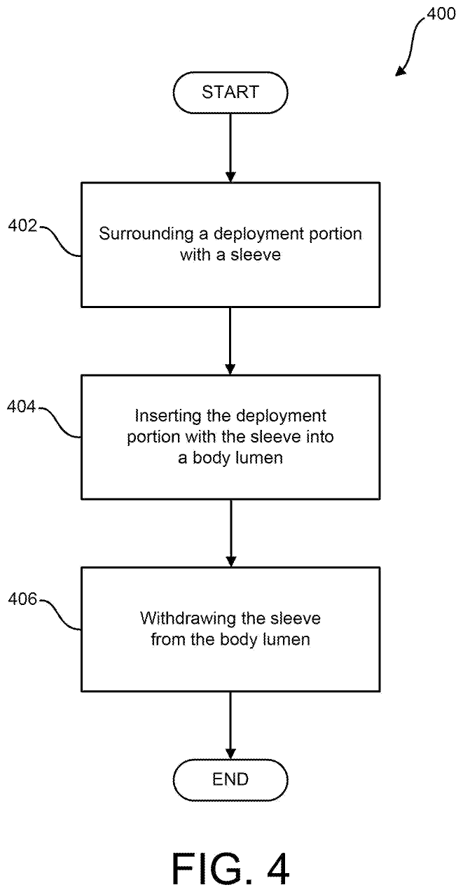

The present disclosure includes methods of delivering a percutaneous medical device. As shown in FIG. 4, such a method 400 can include at step 402 surrounding a deployment portion of the percutaneous medical device with a delivery device. The delivery device can be similar to those disclosed elsewhere herein, including the delivery device 210. For example, the delivery device can include an enveloper member and a tether assembly. The enveloper member can include an enveloper wall and an enveloper aperture formed at the enveloper wall such that the enveloper member is configured to receive a deployment portion of the percutaneous medical device through the enveloper aperture. The tether assembly can extend from the enveloper wall. The enveloper member can be configured to removably cover the deployment portion with the enveloper wall. The tether assembly can be configured to facilitate withdrawal of the enveloper member from covering the deployment portion and to facilitate withdrawal of the enveloper member from a delivery lumen. The method 400 can include at step 404 inserting the deployment portion together with the enveloper member into a body lumen. The method 400 can include at step 406 withdrawing the enveloper member from the body lumen.

Examples of the method 400 can include a multi-step insertion process. In examples, the method 400 can include withdrawing the deployment portion together with the enveloper member into a transfer tube. In examples, inserting the deployment portion can include inserting the transfer tube into the body lumen and moving the deployment portion distally relative to the transfer tube such that the deployment portion is outside of the transfer tube.

In examples, as noted above, a distal portion of the enveloper member can include a securement member that is configured to inhibit expansion of the distal portion of the enveloper member when the securement member is secured and to allow the distal portion of the enveloper member to expand when the securement member is released. The tether assembly can be configured to cause the securement member to transition from a secured state to an unsecured state. Inserting the deployment portion together with the enveloper member into the body lumen can occur with the enveloper member in the secured state. The method 400 can include causing a portion of the enveloper member to transition from the secured state to the unsecured state.

In examples, as noted above, the tether assembly can include a collar that connects the tether assembly to the enveloper member at a proximal end of the enveloper member. The enveloper aperture can be distally positioned relative to the collar.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

While the present disclosure has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practices in the art to which this invention pertains.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A delivery device for percutaneous medical device, the delivery device comprising: an enveloper member that includes an enveloper wall, an enveloper aperture that is formed at the enveloper wall such that the enveloper member is configured to receive a deployment portion of the percutaneous medical device through the enveloper aperture, and a tether assembly extending from the enveloper wall; the enveloper member configured to removably cover the deployment portion with the enveloper wall; and the tether assembly configured to facilitate removal of the enveloper member from covering the deployment portion and to facilitate withdrawal of the enveloper member from a delivery lumen.

2. The delivery device of claim 1, wherein the tether assembly is fixedly connected to the enveloper member.

3. The delivery device of claim 1, wherein the tether assembly includes a collar that connects the tether assembly to the enveloper member.

4. The delivery device of claim 3, wherein the collar is configured to inhibit motion of the enveloper member relative to the percutaneous medical device.

5. The delivery device of claim 1, wherein a distal portion of the enveloper member includes a securement member that is configured to inhibit expansion of the distal portion of the enveloper member when the securement member is secured and to allow the distal portion of the enveloper member to expand when the securement member is released.

6. The delivery device of claim 5, wherein the enveloper member is a braided mesh enveloper member.

7. The delivery device of claim 6, wherein the securement member comprises silicone.

8. The delivery device of claim 1, wherein the enveloper member comprises nitinol.

9. A delivery assembly for a percutaneous medical device, the delivery assembly comprising:
a transfer tube having an inner lumen that is configured to removably house a deployment portion of the percutaneous medical device; and
a delivery device comprising: an enveloper member that includes an enveloper wall, an enveloper aperture that is formed at the enveloper wall such that the enveloper member is configured to receive a deployment portion of the percutaneous medical device through the enveloper aperture, and a tether assembly extending from the enveloper wall, the enveloper member configured to removably cover the deployment portion with the enveloper wall, and the tether assembly configured to facilitate withdrawal of the enveloper member from covering the deployment portion and to facilitate withdrawal of the enveloper member from a delivery lumen.

10. The delivery assembly of claim 9, wherein the tether assembly is fixedly connected to the enveloper member.

11. The delivery assembly of claim 9, wherein the tether assembly includes a collar that connects the tether assembly to the enveloper member at a proximal end of the enveloper member.

12. The delivery assembly of claim 1, wherein the collar is configured to inhibit motion of the enveloper member relative to the percutaneous medical device.

13. The delivery assembly of claim 9, wherein a distal portion of the enveloper member includes a securement member that is configured to inhibit expansion of the distal portion of the enveloper member when the securement member is secured and to allow the distal portion of the enveloper member to expand when the securement member is released.

14. The delivery assembly of claim 13, wherein the enveloper member is a braided mesh enveloper member.

15. The delivery assembly of claim 14, wherein the securement member comprises silicone.

16. The delivery assembly of claim 9, wherein the enveloper member comprises nitinol.

17. A method of delivering a percutaneous medical device, the method comprising:
surrounding a deployment portion of the percutaneous medical device with a delivery device comprising: an enveloper member that includes an enveloper wall, an enveloper aperture that is formed at the enveloper wall such that the enveloper member is configured to receive a deployment portion of the percutaneous medical device through the enveloper aperture, the enveloper member configured to removably cover the deployment portion with the enveloper wall, and the tether assembly configured to facilitate withdrawal of the enveloper member from covering the deployment portion and to facilitate withdrawal of the enveloper member from a delivery lumen;
inserting the deployment portion together with the enveloper member into a body lumen; and
withdrawing the enveloper member from the body lumen.

18. The method of claim 17 further comprising withdrawing the deployment portion together with the enveloper member into a transfer tube, and wherein inserting the deployment portion includes inserting the transfer tube into the body lumen and moving the deployment portion distally relative to the transfer tube such that the deployment portion is outside of the transfer tube.

19. The method of claim 17, wherein a distal portion of the enveloper member includes a securement member that is configured to inhibit expansion of the distal portion of the enveloper member when the securement member is secured and to allow the distal portion of the enveloper member to expand when the securement member is released, and wherein the tether assembly is configured to cause the securement member to transition from a secured state to a unsecured state, wherein inserting the deployment portion together with the enveloper member into the body lumen occurs with the enveloper member in the secured state, and wherein the method further comprises causing a portion of the enveloper member to transition from the secured state to the unsecured state.

20. The method of claim 19, wherein the tether assembly includes a collar that connects the tether assembly to the enveloper member at a proximal end of the enveloper member, and wherein the enveloper aperture is distally positioned relative to the collar.
